(19) United States
(12) Patent Application Publication
(10) Pub. No.: US 2021/0322256 A1
(43) Pub. Date: Oct. 21, 2021

(54) EXTERNAL COUNTERPULSATION SYSTEM AND METHOD FOR CONTROLLING SAME

(71) Applicant: OSTEOSYS CO., LTD., Seoul (KR)

(72) Inventors: Young Bok AHN, Seoul (KR); Won Hee LEE, Anyang-si Gyeonggi-do (KR); Jae Jin KIM, Seoul (KR)

(73) Assignee: OSTEOSYS CO., LTD., Seoul (KR)

(21) Appl. No.: 16/622,221

(22) PCT Filed: Jul. 10, 2018
(86) PCT No.: PCT/KR2018/007774

§ 371 Date: Dec. 12, 2019

(30) Foreign Application Priority Data
Feb. 13, 2018 (KR) 10-2018-0017453

(51) Int. Cl. A61H 9/00

(52) U.S. Cl. CPC A61H 9/0007; A61H 9/0092; A61H 2201/164; A61H 2201/1207; A61H 2201/1238; A61H 2230/255; A61H 2201/5048; A61H 2205/10; A61H 2230/045; A61H 2230/065; A61H 2201/1635

(57) ABSTRACT

The present invention relates to an external counterpulsation system which senses a biosignal to monitor a heart condition and performs external counterpulsation in response to the monitored heart condition. The present invention comprises: a compression device surrounding limbs of a subject to compress the surrounded limbs or release the limbs from the compression; and a control device which controls the compression device to apply test compression to the limbs for a predetermined test period with predetermined test intensity before performing the external counterpulsation, configures personalized maximum compression intensity corresponding to the subject through the test compression, and controls the compression device to perform the external counterpulsation on the basis of the configured maximum compression intensity.

### TECHNICAL FIELD

The present invention relates to an external counterpulsation system that repeatedly compresses or decompresses limbs on the basis of an electrocardiogram, thus improving cardiovascular disease, and to a method of controlling the same.

### BACKGROUND ART

An external counterpulsation system is used to perform non-invasive, safe, low-cost, and high efficiency treatment for ischemic heart disease. The external counterpulsation system is a system that treats symptoms such as angina, myocardial infarction, and the like without surgical intervention by increasing the blood flow of dormant blood vessels. The external counterpulsation system compresses or decompresses limbs according to cardiac cycle.

The external counterpulsation system involves the use of external compressive cuffs placed around a patient's lower limbs. While the patient lies on a table with the cuffs placed around his or her calves, thighs, and buttocks, the cuffs inflate during diastole and deflate right before systole. These inflations and deflations repeat regularly in synchronization with a patient's cardiac cycle.

One example of the external counterpulsation system may include a controller, a pneumatic compressor, a set of solenoid valves, and an inflation device. The controller performs treatment by controlling inflation/deflation of the inflation device through control of the pneumatic compressor and the set of solenoid valves according to a sensed cardiac cycle. As such, the pneumatic compressor is typically used for inflation of the inflation device.

However, due to the use of the pneumatic compressor to inflate the inflation device such as balloon cuff, the external counterpulsation system in the related art as above is problematic in that undesired noise and vibration are generated. In addition, the external counterpulsation system in the related art is configured to manually set maximum compression intensity for the patient.

However, such manual setting of the maximum compression intensity requires the presence of an operator who sets the maximum compression intensity, causing inconvenience. In addition, there is a risk that an error may occur during the manual setting by the operator.

### DISCLOSURE

### Technical Problem

Accordingly, the present invention has been made keeping in mind the above problems occurring in the related art, and an objective of the present invention is to provide an external counterpulsation system that automatically configures initial compression intensity without requiring a manual configuring process and reduces noise and vibration problems occurring in a conventional pneumatic drive method, and to provide a method of controlling the same.

### Technical Solution

In order to accomplish the above objective, according to one aspect of the present invention, there is provided an external counterpulsation system that senses biosignals to monitor a heart condition and performs external counterpulsation on the basis of the monitored heart condition.

The external counterpulsation system may include: a compression device wrapping around limbs of a subject to compress or decompress the wrapped limbs; and a controller configured to control the compression device to apply test compression to the limbs for a predetermined test period with predetermined test intensity before performing the external counterpulsation, configure personalized maximum compression intensity specified to the subject through the test compression, and control the compression device to perform the external counterpulsation on the basis of the configured maximum compression intensity.

The controller may perform the test compression while incrementally increasing the test intensity for the predetermined test period, and configure the maximum compression intensity on the basis of the increased test intensity when compression due to the increased test intensity is determined to exceed an acceptable compression range which is an acceptable range to the subject.

Herein, the controller may configure any one of test intensities at levels before the test intensity is increased as the maximum compression intensity when compression due to the incrementally increased test intensity is determined to exceed the acceptable compression range.

The external counterpulsation system may further include a user input device generating, when pain occurs in the subject due to the compression of the limbs during the test compression and an operator or subject's intention is input in association with the pain, a notification signal associated with occurrence of the pain and outputting the notification signal to the controller, wherein the controller may determine that compression due to the increased test intensity exceeds the acceptable compression range when the notification signal is received from the user input device.

Herein, the user input device may sense and recognize an operator or subject's voice during the test compression, determine that the pain has occurred in the subject when a recognized voice signal corresponds to a predetermined reference voice signal, and generate the notification signal to output the notification signal to the controller.

The external counterpulsation system may further include a user input device sensing and recognizing an operator or subject's voice during the test compression, determining that pain has occurred in the subject when a recognized voice signal corresponds to a predetermined reference voice signal, and generating a notification signal associated with occurrence of the pain to output the notification signal to the controller, wherein the controller may determine that compression due to the increased test intensity exceeds the acceptable compression range when the notification signal is received from the user input device.

The external counterpulsation system may further include a biosignal sensing device measuring the biosignals of the subject during the test compression and outputting the biosignals to the controller, wherein the controller may determine that compression due to the increased test intensity exceeds the acceptable compression range when the biosignals received from the biosignal sensing device correspond to predetermined stress reference values.

The biosignal sensing device may include an electrocardiogram measuring unit measuring an electrocardiogram of the subject, and a blood flow rate measuring unit measuring a blood flow rate of the subject, wherein the controller may determine that compression due to the increased test intensity exceeds the acceptable compression range when the electrocardiogram and the blood flow rate respectively measured by the electrocardiogram measuring unit and the blood flow rate measuring unit reach predetermined stress reference values during the test compression.

The controller may configure an initial value of the test intensity within a range of 0.095 to 0.122 kgf/cm².

The controller may analyze an electrocardiogram and a blood flow rate among the biosignals to calculate a compression/decompression timing control signal for the external counterpulsation on the basis of the maximum compression intensity configured through the test compression, and control the compression device on the basis of the calculated timing control signal.

The compression device may include a cuff unit wrapping around the limbs, and a drive unit electrically driving the cuff unit to allow the limbs to be compressed or decompressed.

According to another aspect of the present invention, there is provided a method of controlling an external counterpulsation system that senses biosignals to monitor a heart condition and performs external counterpulsation on the basis of the monitored heart condition by controlling a compression device wrapping around limbs of a subject to compress and decompress the wrapped limbs.

The method may include: controlling the compression device to apply test compression to the limbs for a predetermined test period with predetermined test intensity before performing the external counterpulsation, and configuring personalized maximum compression intensity specified to the subject through the test compression; and controlling the compression device to perform the external counterpulsation on the basis of the configured maximum compression intensity.

The configuring the personalized maximum compression intensity specified to the subject through the test compression may include: performing the test compression while incrementally increasing the test intensity for the predetermined test period; and configuring the maximum compression intensity on the basis of the increased test intensity when compression due to the increased test intensity is determined to exceed an acceptable compression range which is an acceptable range to the subject.

### Advantageous Effects

As described above, the present invention can automatically configure initial compression intensity through test compression, thus ensuring that, unlike the related art technology, the presence of an operator is not necessarily required and an error that may occur when the initial compression intensity is manually input is avoided. Furthermore, the present invention can configure the initial compression intensity at maximum level to be personalized to a subject, thus ensuring that enhanced therapeutic effect of external counterpulsation is provided.

Furthermore, the present invention can implement a compression device by an electric drive method, thus performing compression and decompression at fine levels compared to a conventional hydraulic drive method during the test compression. This ensures that maximum compression intensity is configured more suitable for the subject easily.

Furthermore, the present invention can implement the compression device for compressing and decompressing limbs by an electric drive method, thus ensuring that noise and vibration are reduced compared to the conventional hydraulic drive method.

### DESCRIPTION OF DRAWINGS

FIG. 1 is a block diagram showing an external counterpulsation system according to an embodiment of the present invention.

FIG. 2 is a block diagram showing a biosignal sensing device according to an embodiment of the present invention.

FIG. 3 is a block diagram showing a user input device according to an embodiment of the present invention.

FIG. 4 is a block diagram showing a compression device according to an embodiment of the present invention.

FIG. 5 is a flowchart showing a method of controlling an external counterpulsation system according to an embodiment of the present invention.

FIG. 6 is a flowchart showing a step of configuring maximum compression intensity according to an embodiment of the present invention.

### BEST MODE

The above and other objectives, features, and advantages of the present invention will be more clearly understood from the following detailed description taken in conjunction with the accompanying drawings, such that the invention can be easily embodied by one of ordinary skill in the art to which this invention belongs. In the following description, it is to be noted that, when the functions of conventional elements and the detailed description of elements related with the present invention may make the gist of the present invention unclear, a detailed description of those elements will be omitted.

It will be understood that, although the terms first, second, etc. may be used herein to describe various elements, these elements should not be limited by these terms. These terms are only used to distinguish one element, from another element. For instance, a first element discussed below could be termed a second element without departing from the teachings of the present invention. Similarly, the second element could also be termed the first element. The terminology used herein is for the purpose of describing particular embodiments only and is not intended to be limiting of example embodiments of the invention. As used herein, the singular forms “a”, “an”, and “the” are intended to include the plural forms as well, unless the context clearly indicates otherwise.

Although terminologies used in the present specification are selected from general terminologies used currently and widely in consideration of functions, they may be changed in accordance with intentions of technicians engaged in the corresponding fields, customs, advents of new technologies, and the like. Occasionally, some terminologies may be arbitrarily selected by the applicant(s). In this case, the meanings of the arbitrarily selected terminologies shall be described in the corresponding part of the detailed description of the specification. Therefore, terminologies used in the present specification need to be construed on the basis of the substantial meanings of the corresponding terminologies and the overall matters disclosed in the present specification rather than construed as simple names of the terminologies.

Unless the context clearly indicates otherwise, it will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Also, the terms “˜ part”, “˜ unit”, “module”, “apparatus”, and the like mean a unit for processing at least one function or operation and may be implemented by a combination of hardware and/or software.

Hereinbelow, exemplary embodiments of the present invention will be described in detail with reference to the accompanying drawings. Various changes to the following embodiments are possible and the scope of the present invention is not limited to the following embodiments. It should be understood that the embodiments of the present invention are presented to make complete disclosure of the present invention and help those who are ordinarily skilled in the art best understand the invention.

Combinations of blocks in the accompanying block diagrams or steps in the accompanying flowcharts can be executed by computer program instructions (execution engine), and the computer program instructions can be mounted in a processor of a general-use computer, special-use computer, or other programmable data processing equipment. Thus, the instructions executed through the processor of the computer or other programmable data processing equipment generate units for performing functions described in the respective blocks of the block diagrams or the respective steps of the flowcharts. The computer program instructions can be stored in a computer usable or readable memory oriented to a computer or other programmable data processing equipment, in order to implement functions in a specific method. Thus, the instructions stored in the computer usable or readable memory can be used to manufacture products including instruction units for performing the functions described in the respective blocks of the block diagrams or the respective steps of the flowcharts.

As described above, the computer program instructions can be mounted in the computer or other programmable data processing equipment. Therefore, instructions which generate processes by performing a series of operation steps on the computer or other programmable data processing equipment and operate the computer or other programmable data processing equipment can provide steps for executing the functions described in the respective blocks of the block diagrams and the respective steps of the flowcharts.

Each of the blocks or steps may indicate a part of a module, segment or code including one or more executable instructions for executing specific logical functions. In some substitutions, the functions described in the blocks or steps can be performed out of sequence. That is, two blocks or steps can be operated or performed substantially at the same time, and the blocks or steps can be operated or performed in the reverse order of the corresponding function.

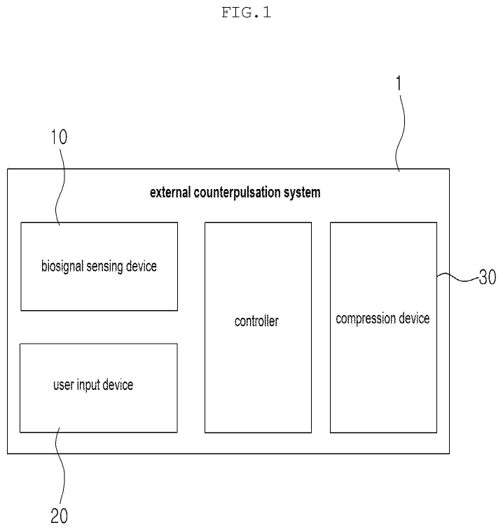

Hereinafter, an external counterpulsation system according to an embodiment of the present invention will be described with reference to FIGS. 1 to 4.

As shown in FIG. 1, an external counterpulsation system 1 according to the present embodiment includes a biosignal sensing device 10, a user input device 20, a compression device 30, and a controller 40 in order to sense a biosignal to monitor a heart condition, and perform external counterpulsation on the basis of the monitored heart condition.

The biosignal sensing device 10 is a device for measuring biosignals of a subject undergoing treatment related to external counterpulsation treatment. The biosignals measured as above are transmitted to the controller 40.

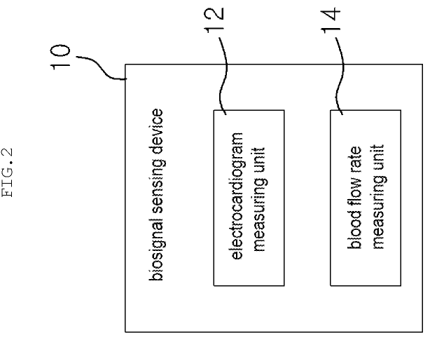

Hereinafter, the biosignal sensing device 10 will be described in detail with reference to FIG. 2.

As shown in FIG. 2, the biosignal sensing device 10 is a device for measuring biosignals of a subject undergoing treatment related to external counterpulsation treatment. The biosignal sensing device may include an electrocardiogram measuring unit 12 and a blood flow rate measuring unit 14.

The biosignal sensing device 10 may further include other biosignal measuring means as necessary in addition to the configuration shown in FIG. 2. The configuration related to the measurement of biosignals is a general known technique, and a detailed description thereof will be omitted.

The electrocardiogram measuring unit 12 measures an electrocardiogram of the subject and transmits the electrocardiogram to the controller 40. The term “electrocardiogram” represents a record of electrical changes that occur locally due to cardiac activity. The electrocardiogram is typically measured by inducing an electric potential by means of electrodes attached to a specific part of the body surface.

The blood flow rate measuring unit 14 is a device for measuring the rate of flow of blood passing through blood vessels in a unit time, and may use a photophethysmography sensor. In addition, the blood flow rate measuring unit 14 may be implemented in various ways, such as an electromagnetic blood flow meter, an ultrasonic blood flow meter, or the like.

In FIG. 2, the biosignal sensing device 10 includes only the electrocardiogram measuring unit 12 and the blood flow rate measuring unit 14. However, this is merely an example, and the biosignal sensing device may further include measuring means for measuring various biosignals, such as a blood pressure measuring unit, a body temperature measuring unit, a skin resistance measuring unit, and the like.

The user input device 20 serves as a user interface that receives information related to operation from a user who can use the present system and delivers the information to the controller 40. Herein, the user is not limited to an operator who performs external counterpulsation treatment through the operation of the system, and the user may include a subject who receives the treatment.

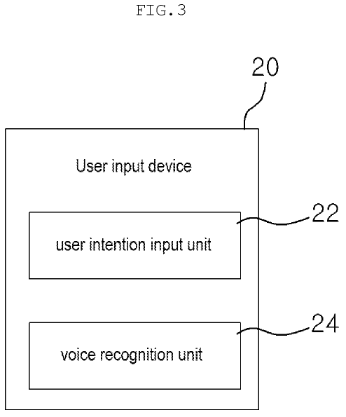

Hereinafter, the user input device 20 will be described in detail with reference to FIG. 3.

Herein, a description of the function of a general user interface such as the operation of the system is omitted, and a description is given only of configurations related to the gist of the present invention.

The user input device 20 may include a user intention input unit 2 receiving a user's intention during test compression and transmitting the user's intention to the controller 40, and a voice recognition unit 24 recognizing user's voice during the test compression and transmitting the user's voice to the controller 40.

The user intention input unit 22 performs a function of, when pain occurs to the subject due to compression of limbs during the test compression, transmitting the occurrence of the pain to the controller 40. The user intention input unit 22 may be implemented in various ways, such as a key button, a touch pad, or the like, to receive an operator or subject's intention when pain occurs in the subject.

The user intention input unit 22 may generate, when a user input indicating the occurrence of pain in the subject is input by the user through a key button or the like, a notification signal corresponding to the input and transmit the notification signal to the controller 40 in a wired or wireless manner.

The voice recognition unit 24 senses and recognizes an operator or subject's voice during the test compression, determines that pain has occurred in the subject when a recognized voice signal corresponds to a predetermined reference voice signal, and generates a notification signal associated with the occurrence of the pain to output the notification signal to the controller 40. Voice recognition and voice recognition processing technology for processing the same are well known in the art, and thus a description thereof will be omitted.

The reference voice signal is determined before the operation of the present system, and various voice signals may be configured as the reference voice signal according to an operating environment of the system. The reference voice signal may be configured variously such as “pause”, “suspend”, “stop”, and the like. The reference voice signal needs to be recognized by the operator or the subject before the operation of the system.

The compression device 30 is a device that wraps around the limbs of the subject under the control of the controller 40 and compresses or decompresses the wrapped limbs. The compression device 30 may be comprised of three compression devices, such as a calf compression device, a lower-thigh compression device, and an upper-thigh compression device. However, if necessary, the compression device 30 may be implemented as one or two compression devices, and the wearing position of the compression device 30 may vary.

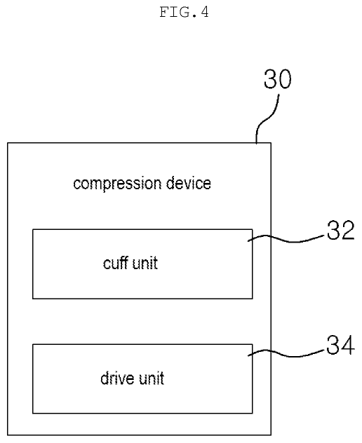

Hereinafter, the compression device 30 will be described in detail with reference to FIG. 4.

As shown in FIG. 4, the compression device 30 may include a cuff unit 32 wrapping around the limbs of the subject, and a drive unit 34 driving the cuff unit 32. The drive unit 34 drives the cuff unit 32 under the control of the control unit 40 to allow the cuff unit to compress or decompress the limbs.

The drive unit 34 may be implemented by an electric drive method, such as an electric motor, a solenoid, or the like. However, if necessary, the drive unit 34 may be implemented by a pneumatic drive method.

When the compression device for compressing and decompressing the limbs is implemented by an electric drive method, there is an advantage of reducing noise and vibration over a conventional hydraulic drive method.

The controller 40 controls the overall operation of the present system and is configured to sense biosignals to monitor a heart condition, and perform external counterpulsation on the basis of the monitored heart condition. In other words, the controller 40 receives signals transmitted from the biosignal sensing device 10 and the user input device 20 and controls the compression device 30 to perform external counterpulsation on the basis of the received signals.

The controller 40 controls the compression device 30 to apply test compression to the limbs for a predetermined test period with predetermined test intensity before performing external counterpulsation and configures personalized maximum compression intensity specified to the subject through the test compression. The controller 40 controls the compression device 30 to perform the external counterpulsation on the basis of the configured maximum compression intensity.

As such, the controller 40 can automatically configure initial compression intensity through the test compression, thus ensuring that, unlike the related art technology, the presence of the operator is not necessarily required and an error that may occur when the initial compression intensity is manually input is avoided.

In addition, the controller 40 can configure the initial compression intensity at maximum level to be personalized to the subject, thus ensuring that enhanced therapeutic effect of the external counterpulsation is provided.

In addition, when the compression device 30 is implemented by an electric drive method, the controller 40 can perform compression and decompression at fine levels compared to the conventional hydraulic drive method during the test compression, thus configuring maximum compression intensity more suitable for the subject precisely and easily.

The controller 40 performs the test compression while incrementally increasing the test intensity for the predetermined test period, and configures the maximum compression intensity on the basis of the increased test intensity when compression due to the increased test intensity is determined to exceed an acceptable compression range which is an acceptable range to the subject.

As an example, the controller 40 may configure any one of test intensities at the levels before the test intensity is increased as the maximum compression intensity when compression due to the incrementally increased test intensity is determined to exceed the acceptable compression range.

When test intensity of the level right before the increased test intensity at the time the acceptable compression range of the subject is exceeded is configured as the maximum compression intensity, the test intensity may also be determined to exceed the acceptable compression range. This is because the acceptable compression range of the subject is typically determined by whether pain occurs in the subject. Therefore, one of test intensities of the levels lower than test intensity of the level right before the increased test intensity may be configured as the maximum compression intensity.

The controller 40 may configure an initial value of test intensity within a range of 0.095 to 0.122 kgf/cm² and configure a maximum limit value of the test intensity within a range of 0.3 to 0.5 kgf/cm², to incrementally increase the test intensity by a value within a range of 0.020 to 0.034 kgf/cm². Herein, the initial value of the test intensity may be configured as 0.109 kgf/cm², the maximum limit value of the test intensity may be configured as 0.5 kgf/cm², and the amount of incremental increase of the test intensity may be configured as 0.027 kgf/cm².

In addition, the controller 40 may control the compression device 30 to perform test compression for 0.5 seconds for a test period of about 1 second and to perform release of the test compression for about 0.5 seconds.

The controller 40 determines that compression due to the increased test intensity exceeds the acceptable compression range which is the acceptable range to the subject when a notification signal associated with pain in the subject from the user input device 20 is received in the process of performing the test compression while incrementally increasing the test intensity for the predetermined test period. In this case, the controller 40 may configure the maximum compression intensity on the basis of the increased test intensity.

In other words, the controller 40 may determine that compression due to the increased test intensity exceeds the acceptable compression range when the notification signal associated with pain in the subject from the user intention input unit 22 is received, and may configure the maximum compression intensity on the basis of the increased test intensity.

In addition, the controller 40 may determine that compression due to the increased test intensity exceeds the acceptable compression range when the notification signal associated with pain in the subject from the voice recognition unit 24 is received, and may configure the maximum compression intensity on the basis of the increased test intensity.

In the present embodiment, the user input device 20 includes the user intention input unit 22 and the voice recognition unit 24 together. However, this is merely an example. The user input device 20 may be implemented by any one of the user intention input unit 22 and the voice recognition unit 24, and may be implemented in various other ways to sense the user's intention.

The controller 40 performs the test compression while incrementally increasing the test intensity for the predetermined test period, determines that compression due to the increased test intensity exceeds the above-mentioned acceptable compression range when the biosignals received from the above-mentioned biosignal sensing device 10 correspond to predetermined stress reference values, and configures the maximum compression intensity on the basis of the increased test intensity.

As an example, the controller 40 may determine that compression due to the increased test intensity exceeds the acceptable compression range when the electrocardiogram and blood flow rate respectively measured by the electrocardiogram measuring unit 12 and the blood flow rate measuring unit 14 reach predetermined stress reference values during the above-mentioned test compression, and may configure the maximum compression intensity on the basis of the increased test intensity.

The controller 40 analyzes biosignals, such as electrocardiogram and blood flow rate, transmitted from the biosignal sensing device 10 to calculate a timing control signal on the basis of the maximum compression intensity configured as above such that compression and decompression of the limbs for the external counterpulsation are performed. The controller 40 controls the compression device 30 to be driven in response to the calculated timing control signal.

The timing control signal for compression and decompression is calculated on the basis of electrocardiogram and blood flow rate signals such that the aortic pressure is increased to a predetermined level at a predetermined time of diastole and the aortic pressure is reduced to a predetermined level at a predetermined time of systole.

When the compression device 30 is comprised of three compression devices, such as a calf compression device, a lower-thigh compression device, and an upper-thigh compression device, the controller 40 calculates the timing control signal such that compression is sequentially performed from the calf compression device, the lower-thigh compression device, and the upper-thigh compression device, allowing the blood flow to be moved from the ends toward the heart. In addition, the controller 40 calculates the timing control signal such that decompression is simultaneously performed in all the compression devices 30. This ensures that the burden on the heart is reduced.

The controller 40 controls the compression device on the basis of the timing control signal calculated as above to compress or decompress the limbs. In detail, the controller 40 transmits a timing control signal to the compression device 30, and the compression device compresses and decompresses the limbs in response to the timing control signal transmitted from the controller 40. The drive unit 34 may drive the cuff unit 32 wrapping around the limbs in response to the transmitted timing control signal to allow the limbs to be compressed and decompressed.

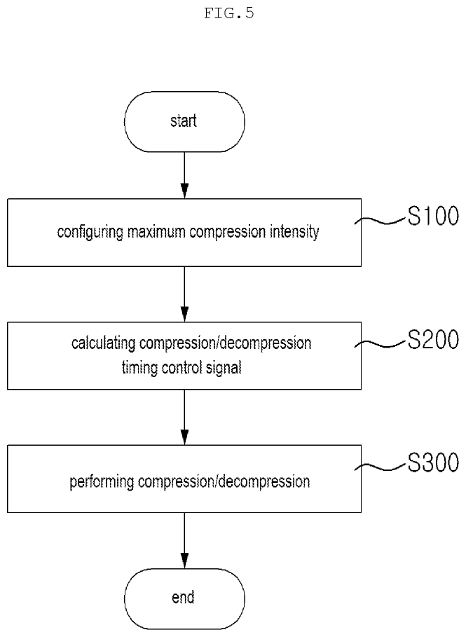

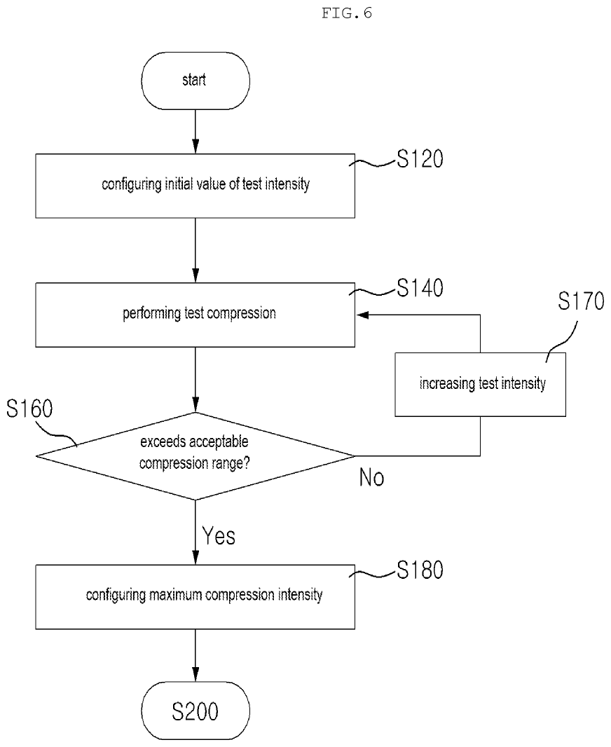

Hereinafter, a method of controlling an external counterpulsation system according to an embodiment of the present invention will be described with reference to FIGS. 5 and 6.

As shown in FIG. 5, a method of controlling an external counterpulsation system includes a maximum compression intensity configuring step (S100), a compression/decompression timing control signal calculation step (S200), and a compression/decompression performing step (S300). The method of controlling the external counterpulsation system 1 may be performed by a controller 40.

First, as in step S100, the external counterpulsation system 1 controls a compression device 30 to apply test compression to the limbs of a subject for a predetermined test period with predetermined test intensity before performing external counterpulsation and configures personalized maximum compression intensity specified to the subject through the test compression.

In other words, the external counterpulsation system 1 performs the test compression while incrementally increasing the test intensity for the predetermined test period, and configures maximum compression intensity on the basis of the increased test intensity when compression due to the increased test intensity is determined to exceed an acceptable compression range which is an acceptable range to the subject.

Next, the external counterpulsation system 1 analyzes biosignals, such as electrocardiogram and blood flow rate, transmitted from a biosignal sensing device 10 to calculate a timing control signal on the basis of the maximum compression intensity configured in step S100 such that compression and decompression of the limbs for the external counterpulsation are performed.

As an example, the timing control signal for compression and decompression may be calculated on the basis of electrocardiogram and blood flow rate signals such that the aortic pressure is increased to a predetermined level at a predetermined time of diastole and the aortic pressure is reduced to a predetermined level at a predetermined time of systole.

Next, the external counterpulsation system 1 controls a compression device 30 on the basis of the timing control signal calculated in step S200 to compress and decompress the limbs (S300).

Hereinafter, the maximum compression intensity configuring step (S100) will be described in detail with reference to FIG. 6.

The external counterpulsation system 1 first configures an initial value of the test intensity (S120) and performs the test compression on the basis of the configured initial value of the test intensity (S140). Herein, the initial value of the test intensity may be configured within a range of 0.095 to 0.122 kgf/cm², and a maximum limit value of the test intensity may be configured within a range of 0.3 to 0.5 kgf/cm².

Next, the external counterpulsation system 1 determines whether the test compression performed in step S140 exceeds the acceptable compression range of the subject (S160).

The external counterpulsation system 1 may determine whether the test compression exceeds the acceptable compression range, on the basis of whether a notification signal associated with pain in the subject is received from a user input device 20 or whether the biosignals received from the biosignal sensing device 10 correspond to stress reference values.

When the result of determination in step S160 is that the test compression of step S140 does not exceed the acceptable compression range, the external counterpulsation system 1 increases the test intensity by a predetermined value (S170), and step S140 is followed. As an example, the external counterpulsation system 1 may configure the amount of increase of the test intensity as about 0.027 kgf/cm². This amount of increase may be configured differently depending on the requirements.

When the result of determination in step S160 is that the test compression of step S140 exceeds the acceptable compression range, the external counterpulsation system 1 configures the maximum compression intensity on the basis of the test intensity used for the test compression of step S140 above (S180).

As an example, when it is determined that the test compression due to the increased test intensity exceeds the acceptable compression range, any one of test intensities at the levels before the test intensity is increased may be configured as the maximum compression intensity.

When it is determined that the test compression by the initial value of the test intensity exceeds the acceptable compression range, the maximum compression intensity may be configured as a value obtained by subtracting a predetermined value from the initial value of the test intensity.

As described above, the external counterpulsation system and the method of controlling the same are characterized by automatically configuring initial compression intensity through the test compression. This ensures that, unlike the related art technology, the presence of the operator is not necessarily required and an error that may occur when the initial compression intensity is manually input is avoided. Furthermore, the present invention can configure the initial compression intensity at maximum level to be personalized to a subject, thus ensuring that enhanced therapeutic effect of the external counterpulsation is provided.

Furthermore, the external counterpulsation system and the method of controlling the same are characterized by implementing a compression device for compression and decompression of the limbs by an electric drive method. This provides an advantage of reducing noise and vibration over a conventional hydraulic drive method.

### INDUSTRIAL APPLICABILITY

The present invention can find wide application in an external counterpulsation system.

1. An external counterpulsation system that senses biosignals to monitor a heart condition and performs external counterpulsation on the basis of the monitored heart condition, the external counterpulsation system comprising:
a compression device wrapping around limbs of a subject to compress or decompress the wrapped limbs; and
a controller configured to control the compression device to apply test compression to the limbs for a predetermined test period with predetermined test intensity before performing the external counterpulsation, configure personalized maximum compression intensity specified to the subject through the test compression, and control the compression device to perform the external counterpulsation on the basis of the configured maximum compression intensity.

2. The external counterpulsation system of claim 1, wherein the controller performs the test compression while incrementally increasing the test intensity for the predetermined test period, and configures the maximum compression intensity on the basis of the increased test intensity when compression due to the increased test intensity is determined to exceed an acceptable compression range which is an acceptable range to the subject.

3. The external counterpulsation system of claim 2, wherein the controller configures any one of test intensities at levels before the test intensity is increased as the maximum compression intensity when compression due to the incrementally increased test intensity is determined to exceed the acceptable compression range.

4. The external counterpulsation system of claim 2, further comprising:
a user input device generating, when pain occurs in the subject due to the compression of the limbs during the test compression and an operator or subject's intention is input in association with the pain, a notification signal associated with occurrence of the pain and outputting the notification signal to the controller, wherein
the controller determines that compression due to the increased test intensity exceeds the acceptable compression range when the notification signal is received from the user input device.

5. The external counterpulsation system of claim 4, wherein the user input device senses and recognizes an operator or subject's voice during the test compression, determines that the pain has occurred in the subject when a recognized voice signal corresponds to a predetermined reference voice signal, and generates the notification signal to output the notification signal to the controller.

6. The external counterpulsation system of claim 2, further comprising:
a user input device sensing and recognizing an operator or subject's voice during the test compression, determining that pain has occurred in the subject when a recognized voice signal corresponds to a predetermined reference voice signal, and generating a notification signal associated with occurrence of the pain to output the notification signal to the controller, wherein
the controller determines that compression due to the increased test intensity exceeds the acceptable compression range when the notification signal is received from the user input device.

7. The external counterpulsation system of claim 2, further comprising:
a biosignal sensing device measuring the biosignals of the subject during the test compression and outputting the biosignals to the controller, wherein
the controller determines that compression due to the increased test intensity exceeds the acceptable compression range when the biosignals received from the biosignal sensing device corresponds to predetermined stress reference values.

8. The external counterpulsation system of claim 7, wherein the biosignal sensing device includes an electrocardiogram measuring unit measuring an electrocardiogram of the subject, and a blood flow rate measuring unit measuring a blood flow rate of the subject, wherein
the controller determines that compression due to the increased test intensity exceeds the acceptable compression range when the electrocardiogram and the blood flow rate respectively measured by the electrocardiogram measuring unit and the blood flow rate measuring unit reach predetermined stress reference values during the test compression.

9. The external counterpulsation system of claim 2, wherein the controller configures an initial value of the test intensity within a range of 0.095 to 0.122 kgf/cm².

10. The external counterpulsation system of claim 1, wherein the controller analyzes an electrocardiogram and a blood flow rate among the biosignals to calculate a compression/decompression timing control signal for the external counterpulsation on the basis of the maximum compression intensity configured through the test compression, and controls the compression device on the basis of the calculated timing control signal.

11. The external counterpulsation system of claim 1, wherein the compression device includes a cuff unit wrapping around the limbs, and a drive unit electrically driving the cuff unit to allow the limbs to be compressed or decompressed.

12. A method of controlling an external counterpulsation system that senses biosignals to monitor a heart condition and performs external counterpulsation on the basis of the monitored heart condition by controlling a compression device wrapping around limbs of a subject to compress and decompress the wrapped limbs, the method comprising:
controlling the compression device to apply test compression to the limbs for a predetermined test period with predetermined test intensity before performing the external counterpulsation, and configuring personalized maximum compression intensity specified to the subject through the test compression; and
controlling the compression device to perform the external counterpulsation on the basis of the configured maximum compression intensity.

13. The method of claim 12, wherein the configuring the personalized maximum compression intensity specified to the subject through the test compression includes:
performing the test compression while incrementally increasing the test intensity for the predetermined test period; and
configuring the maximum compression intensity on the basis of the increased test intensity when compression due to the increased test intensity is determined to exceed an acceptable compression range which is an acceptable range to the subject.
